(19) United States
(12) Patent Application Publication
(10) Pub. No.: US 2007/0254303 A1
(43) Pub. Date: Nov. 1, 2007

(54) Methods for assessing the efficacy of treatment with a glucocorticoid

(75) Inventors: Gerhard Hannig, Revere, MA (US); William F. Westlin, Boxborough, MA (US)

Correspondence Address: LAHIVE & COCKFIELD, LLP, ONE POST OFFICE SQUARE, BOSTON, MA, 02109-2127, US

(73) Assignee: Praecis Pharmaceuticals Incorporated, Waltham, MA (US)

(21) Appl. No.: 11/787,848

(22) Filed: Apr. 18, 2007

Related U.S. Application Data
(60) Provisional application No. 60/792,994, filed on Apr. 18, 2006.

(51) Int. Cl. C12Q 1/68; G01N 33/53

(52) U.S. Cl. 435/6; 435/7.5

(57) ABSTRACT

The invention relates to methods for assessing the efficacy of treatment with a glucocorticoid by determining the amount of MetAP-2.

### RELATED APPLICATION

This application claims priority to U.S. Provisional Application No. 60/792,994, filed Apr. 18, 2006, the entire contents of which are incorporated herein by this reference.

### BACKGROUND OF THE INVENTION

Glucocorticoids are among the most potent and widely used immunosuppressant/immunoregulatory drugs available. Glucocorticoids are commonly used to treat a wide range of diseases, disorders and conditions such as short-term inflammation due to, for example, orthopedic injury, infectious disease, organ transplant, spinal cord injury, chronic autoimmune and inflammatory diseases, and certain eye diseases. In addition, glucocorticoids are a mainstay of dermatology used to combat psoriasis, eczema, acne, dandruff, poison ivy, and other allergic rashes. However, the long-term use of glucocorticoid for chronic diseases, disorders, and conditions is associated with detrimental side effects such as, central obesity, hypertension, impaired wound healing, increased infection rates, bone weakening and osteoporosis, cataracts, and impaired growth in children. In many cases, patients must be tapered off the drug very slowly to prevent a new flare-up and/or an inability of the adrenal glands to respond to stress. Furthermore, a significant number of subjects do not respond to treatment with glucocorticoids, become refractory to prolonged treatment with glucocorticoids, or have treatment interrupted due to intolerable side effects. Accordingly, there is an urgent need in the field for better indicators to guide the vigor and extent of treatment with glucocorticoids.

### SUMMARY OF THE INVENTION

The present invention provides methods and kits for assessing the efficacy of a glucocorticoid treatment for treating a glucocorticoid-associated disease, disorder, or condition in a subject. The present invention is based, at least in part, on the discovery that treatment of a subject with a glucocorticoid, such as for example, dexamethasone, results in a decrease in the amount of methionine aminopeptidase 2 (MetAP-2) protein.

In one aspect, the invention provides a method of assessing the efficacy of a glucocorticoid treatment for treating a subject, e.g., a human, with a glucocorticoid-associated disease, disorder, or condition. The method includes comparing the amount of MetAP-2, e.g., free MetAP-2 protein or MetAP-2 mRNA, in a first sample obtained from the subject prior to administering at least a portion of the glucocorticoid treatment to the subject; and the amount of MetAP-2 in a second sample obtained from the subject following administration of at least a portion of the glucocorticoid treatment, thereby assessing the efficacy of a glucocorticoid treatment for treating a subject with a glucocorticoid-associated disease, disorder, or condition. In one embodiment, a higher amount of MetAP-2 in the first sample, relative to the second sample, is an indication that the glucocorticoid treatment is efficacious for treating a glucocorticoid-associated disease, and a lower amount of MetAP-2 in the first sample, relative to the second sample, is an indication that the treatment regimen is not efficacious for treating a glucocorticoid-associated disease in the subject. The amount of MetAP-2 that is determined in the methods of the invention may be free MetAP-2 protein.

In one embodiment of the invention, determining the amount of MetAP-2 comprises the use of a MetAP-2 inhibitor, such as a biotinylated MetAP-2 inhibitor. In one embodiment, the MetAP-2 inhibitor is a compound comprising the structure

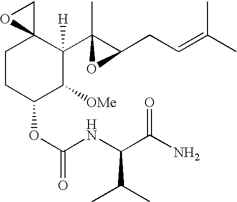

In another aspect, the invention provides a method of assessing the efficacy of a glucocorticoid treatment for treating a glucocorticoid-associated disease in a subject, e.g., a human. The method involves comparing the expression and/or activity of MetAP-2 in a first sample obtained from the subject prior to administering at least a portion of the glucocorticoid treatment to the subject and the expression and/or activity of MetAP-2 in a second sample obtained from the subject following administration of at least a portion of the glucocorticoid treatment, thereby assessing the efficacy of a glucocorticoid treatment for treating a glucocorticoid-associated disease in a subject. A higher expression and/or activity of MetAP-2 in the first sample, relative to the second sample, is an indication that the glucocorticoid treatment is efficacious for treating a glucocorticoid-associated disease, and a lower expression and/or activity of MetAP-2 in the first sample, relative to the second sample, is an indication that the glucocorticoid treatment is not efficacious for treating a glucocorticoid-associated disease in the subject.

In one embodiment, the expression of MetAP-2 is determined using a technique selected from the group consisting of quantitative PCR and expression array analysis.

In one embodiment, the activity of MetAP-2 is determined by determining the amount of free methionine in the first and second sample.

In one embodiment, the sample is a whole blood sample. In another embodiment, the sample is selected from the group consisting of tissue or cells, e.g., white blood cells.

In one embodiment of the invention, the glucocorticoid is selected from the group consisting of dexamethasone, beclomethasone dipropionate, betamethasone, dipropionatebudesonide, cortisone, deflazacort, flunisolide, fludrocortisone, fluticasone propionate, hydrocortisone, methylprednisolone, prednisolone, and triamcinolone.

In one embodiment, the glucocorticoid-associated disease is selected from the group consisting of transplant rejection, allergic disease, autoimmune disease, and cancer. In one embodiment, the autoimmune disease is rheumatoid arthritis.

In yet another aspect, the invention features a kit for performing the methods of the invention. The kit contains, for example, a MetAP-2 inhibitor, a detectable antibody that specifically binds to MetAP-2, reagents for isolating a sample, e.g., whole blood, from the subject, and instructions for use.

In a further aspect, the invention features a method of treating a subject suffering from a MetAP-2 disease. The method includes selecting a subject that would benefit from a decreased amount of MetAP-2, and administering to the subject a therapeutically effective amount of a glucocorticoid, thereby treating a subject suffering from a MetAP-2 disease.

In another aspect, the invention features a method of treating a subject suffering from a glucocorticoid-associated disease. The method includes selecting a subject that would benefit from treatment with a glucocorticoid, and administering to the subject a therapeutically effective amount of a MetAP-2 inhibitor, thereby treating a subject suffering from a glucocorticoid-associated disease.

Other features and advantages of the invention will be apparent from the following detailed description and claims.

### BRIEF DESCRIPTION OF THE DRAWINGS

FIG. 1 is a graph demonstrating that the inhibition of MetAP-2 in vivo by the MetAP-2 inhibitor is correlated with the suppression of chronic arthritis. After completion of the study, wbc from rats of different groups that had been treated with vehicle (p.o., qod), dex (1 mg/kg, p.o., qod) or MetAP-2 inhibitor (1, 5, or 10 mg/kg, p.o., qod) were pooled, lysed, and the amount of MetAP-2 inhibited in wbc lysates was determined by the MetAP-2 pharmacodynamic assay. All values are expressed as percentage of the amount of MetAP-2 inhibited compared with vehicle-treated rats. The amount of uninhibited MetAP-2 (=100%) in vehicle-treated rats in this assay corresponded to 41 ng MetAP-2 per mg wbc protein. The detection limit was 0.47 ng MetAP-2 per mg wbc protein. Columns: 10 rats per group, except for naive rats treated with vehicle (n=4).

### DETAILED DESCRIPTION OF THE INVENTION

The present invention provides methods and kits for assessing the efficacy of a glucocorticoid treatment for treating a subject with a glucocorticoid-associated disease, disorder, or condition. The present invention is based, at least in part, on the discovery that treatment of a subject with a glucocorticoid, e.g., dexamethasone, results in a decrease in the amount of circulating or free methionine aminopeptidase 2 (MetAP-2) protein.

Although the alteration of the amount of MetAP-2, e.g., free MetAP-2 protein, described herein was identified in rheumatoid arthritis samples treated with dexamethasone, the methods of the invention are in no way limited to use for the assessment of the efficacy of dexamethasone treatment of a subject with rheumatoid arthritis. Rather, the methods of the invention may be applied to the use of any glucocorticoid for the treatment of any glucocorticoid-associated disease as described herein.

Various aspects of the invention are described in further detail in the following subsections:

### I. DEFINITIONS

The articles “a” and “an” are used herein to refer to one or to more than one (i.e. to at least one) of the grammatical object of the article. By way of example, “an element” means one or more elements.

As used herein the term “glucocorticoid” is well known in the art and includes, for example, a synthetic corticosteroid such as dexamethasone, beclomethasone dipropionate, betamethasone, dipropionatebudesonide, cortisone, deflazacort, flunisolide, fludrocortisone, fluticasone propionate, hydrocortisone, methylprednisolone, prednisolone, and triamcinolone.

A “glucocorticoid-associated disease” as used herein, refers to a disease, disorder, or condition that a skilled practitioner would routinely treat with the administration of a glucocorticoid. In one embodiment, a glucocorticoid-associated disease is a disease, disorder, or condition that a skilled practitioner would routinely treat with repeated administration, e.g., more than one administration of a seven day course, of a glucocorticoid. Non-limiting examples of glucocorticoid-associated diseases, disorders, and conditions include, transplant rejection; allergic diseases (e.g., asthma, chronic obstructive pulmonary disease (COPD), eczema, rhinitis, atopic dermatitis and urticaria); and autoimmune diseases (e.g., inflammatory bowel syndrome, type 1 diabetes, rheumatoid arthritis, multiple sclerosis, myasthenia gravis, systemic lupus erythematosis, autoimmune thyroiditis, atopic dermatitis, eczematous dermatitis, psoriasis, Sjogren's Syndrome, vasculitis, alopecia areata, allergic responses due to arthropod bite reactions, Crohn's disease, conjunctivitis, ulcerative colitis, asthma, allergic asthma, cutaneous lupus erythematosus, autoimmune uveitis, idiopathic thrombocytopenia, chronic active hepatitis, lichen planus, Juvenile idiopathic arthritis, alopecia universalis, autoimmune uveitis, autoimmune hemolytic anemia, pernicious anemia (due to autoimmune gastritis), and chromic autoimmune hepatitis); chronic infections such as, for example, tuberculosis, HIV, fungal infections, and parasitic infection, such as an infection by Plasmodium species, such as Plasmodium falciparum, or an infection by Leishmania species, such as Leishmania donavani.

As used herein, a glucocorticoid-associated disease, disorder, or condition also includes cancer. The terms “tumor” or “cancer” are well known in the art and refer to the presence, e.g., in a subject, of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within a subject, or may be non-tumorigenic cancer cells, such as leukemia cells. As used herein, the term “cancer” includes pre-malignant as well as malignant cancers. Cancers include, but are not limited to, breast cancer, prostate cancer, ovarian cancer, gastric cancer, colorectal cancer, skin cancer, e.g., melanomas or basal cell carcinomas, lung cancer, cancers of the head and neck, bronchus cancer, pancreatic cancer, urinary bladder cancer, brain or central nervous system cancer, peripheral nervous system cancer, esophageal cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Cancers also include lymphoid cancers, such as lymphoid leukemias, e.g., chronic lymphoid leukemia and acute lymphoid leukemia, and lymphomas, e.g., Non-Hodgkin's lymphoma, including T cell lymphoma and B cell lymphoma.

As used herein, a “tumor cell” or a “cancer cell” is a cancerous cell within, or originating from, a tumor. Tumor or cancer cells are distinct from other, non-cancerous cells present in a tumor, such as vascular cells.

Additional glucocorticoid-associated diseases, disorders and conditions include drug-induced adrenal insufficiency (due to abrupt cessation of treatment with glucocorticoids or due to treatment with Megestrol, Ketoconazole, Metyrapone, Aminoglutethimide, Mitotane), social and spider phobias, Addison's disease, hypersensitivity pneumonitis, pituitary infarction, hypopituitarism, and hypervitaminosis D that is not medication related.

Methionine aminopeptidase-2, referred to herein as “MetAP-2”, is a metallopeptidase that selectively catalyzes the removal of the initiator methionine residue from nascent polypeptides to produce the active form of the protein, a critical step in protein maturation (Keeling and Doolittle (1986) TIBS 21:285; Arfin, et al. (1995) Proc. Natl. Acad. Sci., USA 92:7714; Bradshaw and Yi (2002) Essays Biochem 38:65). This co-translational processing step is the most frequently occurring protein modification and is important in the regulation of a number of cellular processes, such as protein turnover, protein targeting, and cell proliferation (see, for example, Bernier et al. (2005) J Cell Biochem 95:1191). The nucleotide and amino acid sequences of MetAP-2 are known and can be found in, for example, GenBank Accession No. gi:27597083, the entire contents of which are incorporated herein by this reference.

As used herein, a “MetAP-2 disease” is a disease that would benefit from a decreased amount of MetAP-2. Non-limiting examples of diseases that would benefit from a decreased amount of MetAP-2 include the glucocorticoid-associated diseases described herein.

The amount of MetAP-2 in a first sample, e.g., a cell or tissue, derived from a subject that has not been treated with a glucocorticoid is “increased or decreased” or “higher or lower” than the amount of MetAP-2 in a second sample, e.g., a cell or tissue, derived from a subject that has been treated with a glucocorticoid, if the amount of MetAP-2 in the first sample is greater or less, respectively, than the amount in the second sample, by an amount that is greater than the standard error of the assay employed to assess the amount. The amount of MetAP-2 in a first sample, e.g., a cell or tissue, derived from a subject that has not been treated with a glucocorticoid, can be considered “higher” or “lower” than the amount of MetAP-2 in a second sample, e.g., a cell or tissue, derived from a subject that has been treated with a glucocorticoid, if the amount of MetAP-2 in the first sample is greater or less, respectively, than the amount in the second sample, by an amount that is greater than the standard error of the assay employed to assess the amount.

As used herein, the term “the amount of MetAP-2” includes the amount of MetAP-2 protein, as well as the amount of MetAP-2 mRNA. In a preferred embodiment of the methods of the invention, the amount of MetAP-2 protein present in a sample is determined. Total cellular MetAP-2 protein and/or free MetAP-2 protein levels may be determined.

As used herein, “total cellular MetAP-2 protein” refers to the total amount of MetAP-2 present in a cell. As used herein, “free MetAP-2 protein” refers to the amount of total cellular MetAP-2 that is not bound to or derivatized by, for example, a MetAP-2 inhibitor, and/or a glucocorticoid. The amount of free MetAP-2 protein may be determined using the MetAP-2 pharmacodynamic assay described in the Examples section below.

A “higher level of expression and/or activity” of MetAP-2 refers to an expression level and/or activity in a sample, e.g., a cell or tissue, derived from a subject that has been treated with a glucocorticoid that is greater than the standard error of the assay employed to assess expression and/or activity, and is preferably at least twice, and more preferably three, four, five or ten or more times the expression level and/or activity of MetAP-2 in a sample, e.g., a cell or tissue, derived from a subject that has not been treated with a glucocorticoid.

A “lower level of expression and/or activity” of MetAP-2 refers to an expression level and/or activity in a sample, e.g., a cell or tissue, derived from a subject that has been treated with a glucocorticoid that is greater than the standard error of the assay employed to assess expression and/or activity, and is preferably at least twice, and more preferably three, four, five or ten or more times less than the expression level and/or activity of MetAP-2 in a sample, e.g., a cell or tissue, derived from a subject that has not been treated with a glucocorticoid.

As used herein, “antibody” includes, by way of example, naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

As used herein, a “subject” is any animal, such as a mammal, and includes, without limitation, humans, mice, monkeys, dogs, cats, mice, rats cows, horses, goats, sheep as well as other farm and pet animals.

A kit is any manufacture (e.g., a package or container) comprising at least one reagent, e.g., a probe or antibody, for specifically detecting MetAP-2, e.g., free MetAP-2 protein, MetAP-2 protein, MetAP-2 mRNA, MetAP-2 activity, the manufacture being promoted, distributed, or sold as a unit for performing the methods of the present invention.

### II. USES OF THE INVENTION

The invention provides methods for assessing the efficacy of a treatment with a glucocorticoid. In these methods the amount of MetAP-2 in a first sample not subjected to glucocorticoid treatment and a second sample subjected to at least a portion of glucocorticoid treatment is assessed. In one embodiment, a higher amount of MetAP-2 (e.g., free MetAP-2 protein or MetAP-2 mRNA) in the first sample, relative to the second sample, is an indication that the glucocorticoid treatment is efficacious for treating a glucocorticoid-associated disease. A lower amount of MetAP-2 (e.g., free MetAP-2 protein or MetAP-2 mRNA) present in the first sample, relative to the second sample would be an indication that the glucocorticoid treatment is not efficacious for treating a glucocorticoid-associated disease in the subject.

The present invention also provides methods for assessing the efficacy of a glucocorticoid treatment for treating a glucocorticoid-associated disease, disorder, or condition in a subject by monitoring the expression and/or activity of MetAP-2. In these methods the expression and/or activity of MetAP-2 in a first sample obtained from the subject prior to administering at least a portion of the glucocorticoid treatment is compared to the expression and/or activity of MetAP-2 in a second sample obtained from the subject following administration of at least a portion of the glucocorticoid treatment. A higher expression and/or activity of MetAP-2 in the first sample, relative to the second sample, is an indication that the glucocorticoid treatment is efficacious for treating a glucocorticoid-associated disease. A lower expression and/or activity of MetAP-2 in the first sample, relative to the second sample, is an indication that the glucocorticoid treatment is not efficacious for treating a glucocorticoid-associated disease in the subject.

The methods of the present invention may be practiced in conjunction with any other method used by the skilled practitioner, such as, for example, radiological analysis, e.g., X-ray, MRI, sonography, bone scanning, densitometry, hematological analysis, e.g., complete blood cell count, identification of infectious agents, e.g., viral, fungal, parasitic, and synovial fluid analysis. Immunohistochemical or immunofluorescence detection (and quantitation if appropriate) of any other molecular marker either by itself, in conjunction with other markers, and/or in conjunction with MetAP-2 may also be used. Suitable markers include, for example, rheumatoid factor, anti-nuclear antibodies, cancer antigens, bacterial antigens, viral antigens, PSA, Ki67, Bcl-2, Bcl-xL, phospho-AKT. Other methods would include detection of other markers by in situ PCR, or by extracting tissue and quantitating other markers by real time PCR. PCR is defined as polymerase chain reaction.

In general, it is preferable that the difference between the amount and/or the expression and/or activity of MetAP-2 in a sample from a subject being treated with a glucocorticoid and the amount and/or the expression and/or activity of MetAP-2 in a sample from a subject not treated with a glucocorticoid, is as great as possible. Although this difference can be as small as the limit of detection of the method for determining the amount and/or the expression and/or activity, it is preferred that the difference be at least greater than the standard error of the assessment method, and preferably a difference of at least 1.5-, 2-, 3-, 4-, 5-, 6-, 7-, 8-, 9-, 10-, 15-, 20-, 25-, 50-, 100-, 500-, 1000-fold or greater.

An alteration in the amount and/or the expression and/or activity of MetAP-2 in a sample from a subject may be assessed in a variety of ways. In one embodiment, the amount and/or the expression and/or activity is assessed by comparing the amount and/or the expression and/or activity of MetAP-2 in a sample from a subject not treated with a glucocorticoid to the amount and/or the expression and/or activity of MetAP-2 with the amount in a sample from a subject treated with a glucocorticoid. For example, the amount and/or the expression and/or activity of MetAP-2 may be assessed prior to the treatment of a subject with a glucocorticoid or from archived subject samples, and the like, and this amount may be compared with the amount and/or the expression and/or activity of MetAP-2 following and/or during treatment with a glucocorticoid. Alternatively, and particularly as further information becomes available as a result of routine performance of the methods described herein, population-average values for MetAP-2 amount and/or expression and/or activity in cells not treated with a glucocorticoid may be used.

### III. METHODS FOR OBTAINING SAMPLES AND DETECTING/QUANTITATING METAP-2

Samples useful in the methods of the invention include any tissue, cell, biopsy, or bodily fluid sample that expresses MetAP-2. In one embodiment, a sample may be a tissue, a cell, whole blood, serum, plasma, buccal scrape, saliva, cerebrospinal fluid, urine, stool, or bronchoalveolar lavage. In preferred embodiments, the sample is whole blood, or fractions thereof, e.g., white blood cells, red blood cells, platelets, or plasma.

Body samples may be obtained from a subject by a variety of techniques known in the art including, for example, by the use of a biopsy or by scraping or swabbing an area or by using a needle to aspirate bodily fluids. Methods for collecting various body samples are well known in the art. Methods for collecting whole blood are well known in the art and generally include venipucture. In one embodiment, whole blood is used in the methods of the invention. In another embodiment, a particular part or fraction of whole blood, e.g., white blood cells, is used in the methods of the invention. Various fractions of whole blood can be prepared by methods known in the art, such as centrifugation, use of Ficoll gradients, or the use of, e.g., leukocyte filters, such as leukocyte depletion filters.

Samples suitable for detecting and quantitating MetAP-2 may be fresh, frozen, or fixed according to methods known to one of skill in the art. Suitable tissue samples may be sectioned and placed on a microscope slide for further analyses. Alternatively, solid samples, i.e., tissue samples, may be solubilized and/or homogenized and subsequently analyzed as soluble extracts.

Once the sample is obtained, any method known in the art to be suitable for detecting and quantitating MetAP-2 may be used (either at the nucleic acid or at the protein level). Such methods are well known in the art and include but are not limited to Western blots, Northern blots, Southern blots, immunohistochemistry, ELISA, e.g., amplified ELISA, radioimmunoassay (RIA), immunoprecipitation, immunofluorescence, flow cytometry, immunocytochemistry, mass spectrometrometric analyses, e.g., MALDI-TOF and SELDI-TOF, nucleic acid hybridization techniques, nucleic acid reverse transcription methods, nucleic acid amplification methods, electrophoresis, capillary electrophoresis, high performance liquid chromatography (HPLC), thin layer chromatography (TLC), hyperdiffusion chromatography.

In one embodiment, the amount of MetAP-2 is determined at the protein level. A preferred agent for detecting a MetAP-2 protein is an antibody capable of binding to MetAP-2 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. Anti-MetAP-2 antibodies are well known and available in the art and include, for example, polyclonal MetAP-2 antibodies, available from, for example, Invitrogen™ (Zymed®), such as the CM33 antibody. Other examples of anti-MetAP-2 antibodies include the Abgent antibody (catalogue no. AP2320b).

In one embodiment, the antibody is labeled, e.g. a radio-labeled, chromophore-labeled, fluorophore-labeled, or enzyme-labeled antibody). In another embodiment, an antibody derivative (e.g. an antibody conjugated with a substrate or with the protein or ligand of a protein-ligand pair (e.g. biotin-streptavidin}), or an antibody fragment (e.g., a single-chain antibody, an isolated antibody hypervariable domain, etc.) which binds specifically with a MetAP-2 protein is used.

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibodies or antigens, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In one embodiment, the amount of MetAP-2 protein is detected using, for example, a MetAP-2 inhibitor, e.g., a biotinylayed MetAP-2 inhibitor, that specifically binds MetAP-2, e.g., free MetAP-2. Examples of such inhibitors include those described in, for example, U.S. Pat. No. 6,548,477 B1; U.S. Pat. No. 6,919,307; U.S. Publication No. US-2005-0239878-A1; U.S. Pat. No. 5,135,919; U.S. Pat. No. 5,180,738; U.S. Pat. No. 5,290,807; U.S. Pat. No. 5,648,382; U.S. Pat. No. 5,698,586; U.S. Pat. No. 5,767,293; U.S. Pat. No. 5,789,405 and Bernier, et al., (2004) Proc. Natl. Acad. Sci., USA 101:10768, the contents of each of which are incorporated herein by reference.

In one preferred embodiment, an ELISA assay is used to determine the amount of MetAP-2 protein, e.g., free MetAP-2 protein. Samples are lysed according to methods known in the art and cellular protein is incubated with a MetAP-2 inhibitor. In one embodiment, the inhibitor is a compound comprising the structure (1-Carbamoyl-2-methyl-propyl)-carbamic acid-(3R, 4S, 5S, 6R )-5-methoxy-4-[(2R, 3R) -2-methyl-3-(3-methyl-but-2-enyl)-oxiranyl]-1-oxa-spiro[2.5]oct-6-yl ester. In one embodiment, the MetAP-2 inhibitor is labeled with a detectable substance, such as biotin and subsequently anchored to a solid phase. Subsequent detection of cellular MetAP-2-inhibitor complexes may be determined as described below.

There are many established methods for anchoring assay components to a solid phase. These include, without limitation, immobilization through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of complexes anchored to the solid phase can be accomplished in a number of methods outlined herein. In one embodiment, the detection of the complexes is determined by use of a MetAP-2 antibody, e.g., a MetAP-2 polyclonal antibody, commercially available from, for example, Invitrogen™ (Zymed®). In one embodiment, detection of the anti-MetAP-2 antibody complexes is accomplished with a secondary labeled antibody, e.g., a horeseradish peroxidase-conjugated anti-IgG antibody. Subsequent determination of the amount of MetAP-2 protein, e.g., free MetAP-2 protein, may include the spectrophotometric analysis of the absorption of these complexes and comparison to a standard curve of total cellular MetAP-2 generated using recombinant MetAP-2 prebound to the biotinylated MetAP-2 inhibitor.

In one embodiment, the amount of MetAP-2 that is determined is the amount of free MetAP-2. “Free MetAP-2” refers to the amount of MetAP-2 protein that is not bound to the MetAP-2 inhibitor.

In another embodiment, Western blot (immunoblot) analysis is used to detect and quantify the presence of MetAP-2 in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind MetAP-2. The anti-MetAP-2 antibodies specifically bind to MetAP-2 on the solid support. These antibodies may be directly labeled or alternatively may be subsequently detected using labeled antibodies (e.g., labeled sheep anti-mouse antibodies) that specifically bind to the anti-MetAP-2. In one embodiment, the immunoblotting method for MetAP-2 described by Towbin, et al. (1979) Proc Natl Acad Sci, USA 76: 4350-4, the contents of which are incorporated herein in their entirety by this reference, is used to detect and quantitate MetAP-2 protein.

In another embodiment, MetAP-2 is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled anti-antibody. Alternatively, the labeling agent may be a third moiety, such as another antibody, that specifically binds to the antibody/polypeptide complex.

In one embodiment, the labeling agent is a second human antibody bearing a label. Alternatively, the second antibody may lack a label, but it may, in turn, be bound by a labeled third antibody specific to antibodies of the species from which the second antibody is derived. The second can be modified with a detectable moiety, e.g. as biotin, to which a third labeled molecule can specifically bind, such as enzyme-labeled streptavidin.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of MetAP-2 can take a wide variety of formats well known to those of skill in the art.

Preferred immunoassays for detecting a polypeptide are either competitive or noncompetitive. Noncompetitive immunoassays are assays in which the amount of captured analyte is directly measured. In one preferred “sandwich” assay, for example, the capture agent (anti-peptide antibodies) can be bound directly to a solid substrate where they are immobilized. These immobilized antibodies then capture polypeptide present in the test sample. The polypeptide thus immobilized is then bound by a labeling agent, such as a second human antibody bearing a label.

In competitive assays, the amount of analyte (polypeptide) present in the sample is measured indirectly by measuring the amount of an added (exogenous) analyte (polypeptide) displaced (or competed away) from a capture agent (anti peptide antibody) by the analyte present in the sample. In one competitive assay, a known amount of, in this case, a polypeptide is added to the sample and the sample is then contacted with a capture agent. The amount of polypeptide bound to the antibody is inversely proportional to the concentration of polypeptide present in the sample.

In one embodiment, the antibody is immobilized on a solid substrate. The amount of polypeptide bound to the antibody may be determined either by measuring the amount of polypeptide present in a polypeptide/antibody complex, or alternatively by measuring the amount of remaining uncomplexed polypeptide. The amount of polypeptide may be detected by providing a labeled polypeptide.

The assays of this invention are scored (as positive or negative or quantity of polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

In vivo techniques for detection of MetAP-2 protein include introducing into a subject a labeled antibody directed against the protein. For example, the antibody can be labeled with a radioactive marker whose presence and location in a subject can be detected by standard imaging techniques. Alternatively, a labeled MetAP-2 inhibitor (such as the ones described herein) could be used and its presence and location in a subject can be detected by standard imaging techniques.

In one embodiment of the invention, proteomic methods, e.g., mass spectrometry, are used for detecting and quantitating MetAP-2. For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis Markers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to detect and quantitate the MetAP-2 proteins. Mass spectrometric methods are described in, for example, U.S. Pat. Nos. 5,622,824, 5,605,798 and 5,547,835, the entire contents of each of which are incorporated herein by reference.

In other embodiments, the expression of MetAP-2 is detected at the nucleic acid level. Nucleic acid-based techniques for assessing expression are well known in the art and include, for example, determining the level of MetAP-2 mRNA in a body sample. Many expression detection methods use isolated RNA. Any RNA isolation technique that does not select against the isolation of mRNA can be utilized for the purification of RNA from cells that express MetAP-2 (see, e.g., Ausubel et al., ed., (1987-1999) Current Protocols in Molecular Biology (John Wiley & Sons, New York). Additionally, large numbers of tissue samples can readily be processed using techniques well known to those of skill in the art, such as, for example, the single-step RNA isolation process of Chomczynski (1989, U.S. Pat. No. 4,843,155).

The term “probe” refers to any molecule that is capable of selectively binding to MetAP-2, for example, a MetAP-2 nucleotide transcript or MetAP-2 protein. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated MRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the MetAP-2 mRNA. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to MetAP-2 MRNA or MetAP-2 genomic DNA.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in detecting the level of MetAP-2 mRNA.

An alternative method for determining the level of MetAP-2 mRNA in a sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, MetAP-2 expression is assessed by quantitative fluorogenic RT-PCR (i.e., the TaqMan™ System). Such methods typically utilize pairs of oligonucleotide primers that are specific for MetAP-2. Methods for designing oligonucleotide primers specific for a known sequence are well known in the art.

The expression levels of MetAP-2 mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of MetAP-2 expression may also comprise using nucleic acid probes in solution.

In one embodiment, the amount of MetAP-2 expression is determined as an absolute expression level of the MetAP-2 mRNA present in a sample. As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a non-cancerous sample, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

In one embodiment of the invention, microarrays are used to detect MetAP-2 expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

In yet other embodiments the activity, e.g., enzymatic activity, of MetAP-2 can be determined by, for example determining the amount of free methionine released in an in vitro methionine aminopeptidase enzyme assay (Ben-Bassat, et al. (1987) J. Bacteriol 169:751; Zou, et al. (1995) Mol. Gen. Genetics 246:247-253, the contents of each of which are incorporated herein in their entirety by this reference). Generally, a sample is added to a reaction mixture containing a peptide containing a methionine residue, e.g., Met-Gly-Met, and released methionine is subsequently quantified at different time points (e.g., at 0, 2, 3, and 5 minutes).

### IV. KITS

The invention also provides compositions and kits for performing the methods of the invention. These kits include one or more of the following: an agent capable of detecting MetAP-2, e.g., a detectable MetAP-2 inhibitor that specifically binds to MetAP-2, reagents for obtaining and/or preparing whole blood samples, and instructions for use.

The kits of the invention may optionally comprise additional components useful for performing the methods of the invention. By way of example, the kits may comprise fluids (e.g., SSC buffer) suitable for annealing complementary nucleic acids or for binding an antibody with a protein with which it specifically binds, one or more sample compartments, an instructional material which describes performance of a method of the invention and the appropriate controls/standards.

### V. METHODS OF TREATMENT

The present invention also provides methods of treatment. In one aspect, the present invention provides a method of treating a subject suffering from a MetAP-2 disease. The method includes selecting a subject that would benefit from a decreased amount of MetAP-2, and administering to the subject a therapeutically effective amount of a glucocorticoid, thereby treating a subject suffering from a MetAP-2 disease. Another aspect of the invention provides a method of treating a subject suffering from a glucocorticoid-associated disease. The method includes selecting a subject that would benefit from treatment with a glucocorticoid, and administering to the subject a therapeutically effective amount of a MetAP-2 inhibitor, thereby treating a subject suffering from a glucocorticoid-associated disease.

As used herein, the term “administering” to a subject includes dispensing, delivering or applying a MetAP-2 inhibitor or a glucocorticoid in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

As used herein, the term “therapeutically effective amount” includes an amount effective, at dosages and for periods of time necessary, to achieve the desired result, e.g., sufficient to treat a MetAP-2 disease or a glucocorticoid-associated disease in a subject. An effective amount of a glucocorticoid or a MetP-2 inhibitor, as defined herein may vary according to factors such as the disease state, age, and weight of the subject, and the ability of the glucocorticoid or the MetP-2 inhibitor to elicit a desired response in the subject. Dosage regimens may be adjusted to provide the optimum therapeutic response. An effective amount is also one in which any toxic or detrimental effects (e.g., side effects) of the glucocorticoid or MetP-2 inhibitor are outweighed by the therapeutically beneficial effects.

A therapeutically effective amount of a glucocorticoid or a MetP-2 inhibitor (i.e., an effective dosage) may range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a glucocorticoid or a MetP-2 inhibitor can include a single treatment or, preferably, can include a series of treatments. In one example, a subject is treated with a glucocorticoid or a MetP-2 inhibitor in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a glucocorticoid or a MetP-2 inhibitor used for treatment may increase or decrease over the course of a particular treatment.

The methods of the invention further include administering to a subject a therapeutically effective amount of a glucocorticoid or a MetP-2 inhibitor in combination with another pharmaceutically active compound known to treat a MetAP-2 disease or a glucocorticoid-associated disease, e.g., anti-TNF-α antibodies, e.g., etanercept and infliximab, non-steroidal anti-inflammatory agents (NSAIDs), disease modifying anti-rheumatic drugs (DMARDs), and e.g., chemotherapeutic agents such as Taxol, Paclitaxel, or Actinomycin D. Other pharmaceutically active compounds that may be used can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell New Jersey, Medical Economics Co., the complete contents of which are expressly incorporated herein by reference. The glucocorticoid or the MetP-2 inhibitor and the pharmaceutically active compound may be administered to the subject in the same pharmaceutical composition or in different pharmaceutical compositions (at the same time or at different times).

This invention is further illustrated by the following examples which should not be construed as limiting. The contents of all references, figures, patents and published patent applications cited throughout this application are hereby incorporated by reference.

### EXAMPLES

### Example 1

Methods

Rat model of Peptidoglycan-Polysaccharide (PG-PS) induced arthritis. Female Lewis rats (109-130 g) were received from Charles River Laboratories. Food and water were available ad libitum. PG-PS (25 mg/kg) was injected intraperitoneally on day 1 and responding animals were randomized into treatment groups on day 14. Vehicle (11% HPCD in PBS), dexamethasone and a methionine aminopeptidase 2 inhibitor (1, 5, and 10 mg/kg) were administered orally, every other day. Paw swelling was monitored using a plethysmometer (Stoelting Co., Woodale, Ill.) according to instrument specifications. The volumes of the two hind paws were measured and averaged on day 1, 4, 6, 8, 10, 13, 15, 17, 20, 22, 23, 27, 29 and 31. Ten animals were assigned to each group except the vehicle group and animals which received no PG-PS, but 10 mg/kg of the methionine aminopeptidase 2 inhibitor (n=4). The methionine aminopeptidase 2 inhibitor comprising the structure structure (1-Carbamoyl-2-methyl-propyl)-carbamic acid-(3R, 4S, 5S, 6R )-5-methoxy-4-[(2R, 3R)-2-methyl-3-(3-methyl-but-2-enyl)-oxiranyl]-1-oxa-spiro[2.5]oct-6-yl ester was used in the present studies.

MetAP-2 pharmacodynamic assay. The MetAP-2 assay is an ELISA assay that measures the amount of uninhibited MetAP-2 in cells or tissues which has not been derivitized by prior treatment with a methionine aminopeptidase 2 inhibitor and/or a glucocorticoid. The assay was performed as described in, for example, Bernier, S. G., et al. (2004) Proc. Natl. Acad. Sci. USA 101: 10768-10773 and Bernier, S. G., et al. (2005) J. Cell. Biochem. 95:1191-1203, the entire contents of each of which are incorporated herein by reference. Briefly, white blood cells from animals of each study group were pooled and cell lysates were prepared as described in, for example, Bernier, S. G., et al. (2004) Proc. Natl. Acad. Sci. USA 101: 10768-10773 and Bernier, S. G., et al. (2005) J. Cell. Biochem. 95:1191-1203. Ten μg to twenty μg of white blood cell protein was incubated with a biotinylated analog of the methionine aminopeptidase 2 inhibitor which covalently binds to the catalytic site of MetAP-2 which has not been derivitized or bound by prior treatment with a methionine aminopeptidase 2 inhibitor and/or a glucocorticoid. After a 1 hour incubation period, the biotinylated MetAP-2-inhibitor complex was captured on a plate with immobilized streptavidin (Pierce). After 1 hour, the plates were washed, and the immobilized biotinylated MetAP-2-inhibitor complex was then detected with an anti-MetAP-2 antibody, CM33 (0.5 μg/ml). After 1 hour incubation, horseradish peroxidase-conjugated goat anti-rabbit IgG was added as a secondary antibody and incubated for 1 hour. After several washing steps, 100 μl of TMB substrate [3.5″-5.5″ tetramethylbenzidine and peroxidase solution (1:1), Kirkegaard & Perry Laboratories] was added for 10 minutes. The reaction was stopped by adding 100 μl of 18 M H2SO4. Analysis was performed by determining the absorption of each well at 450 nm by using a Labsystems (Chicago) Multiskan plate spectrophotometer. Human recombinant MetAP-2 (Mediomics), prebound to the biotinylated PPI-2458 analog, was used to generate the standard curve. The detection limit of this assay was 0.47 ng MetAP-2 protein/mg white blood cell protein.

Results

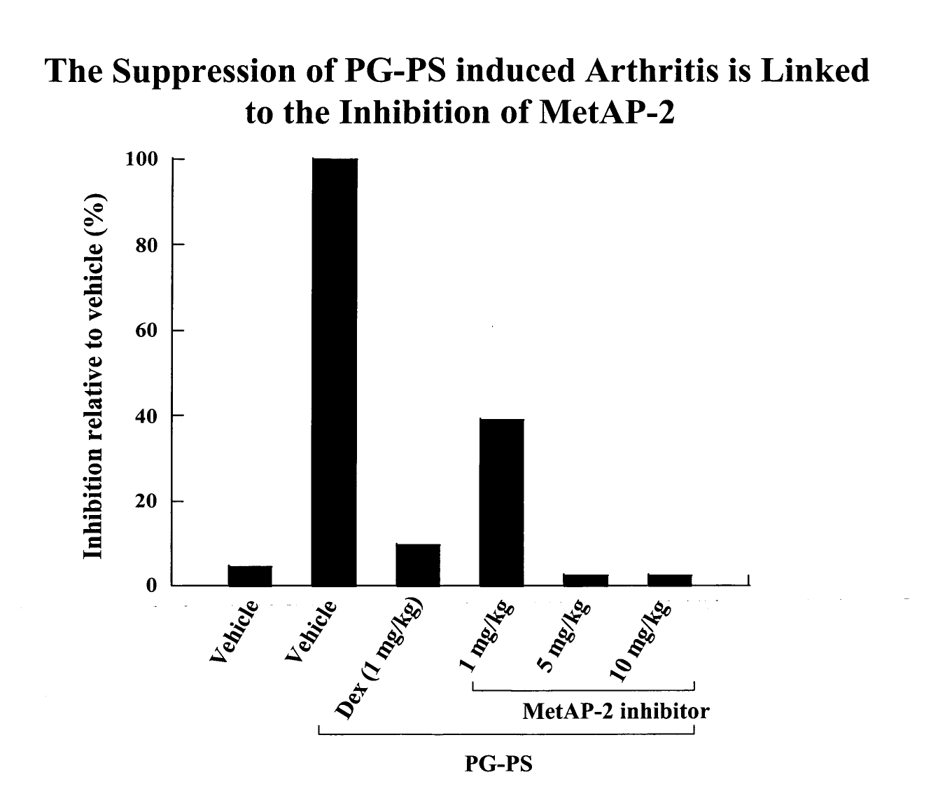

The methionine aminopeptidase 2 inhibitor was shown to inhibit multiple cell types critical for rheumatoid arthritis pathogenesis in vitro and, as such, it was determined whether these observations from in vitro studies would translate into protection from disease in animals in the PG-PS model of arthritis. The progression of disease in this model follows a biphasic mode, with an early acute, predominantly neutrophil-driven phase which persists to days 6-7, followed by a chronic, T cell dependent phase (evident around day 12), which is characterized by chronic inflammation and erosive synovitis (Palombella, V. J., et al. (1998) Proc. Natl. Acad. Sci. USA 95:15671-15676.). Therapeutic dosing of animals administered the methionine aminopeptidase 2 inhibitor orally at 1, 5 and 10 mg/kg, every other day, or vehicle started at day 15 after the chronic destructive phase of the disease was established and terminated on day 31. Consistent with previous results, the methionine aminopeptidase 2 inhibitor at all 3 doses demonstrated significant amelioration of joint swelling and inflammation, measured by paw swelling of the hind limbs, when compared to vehicle-treated animals (Bernier, S. G., et al. (2004) Proc. Natl. Acad. Sci. USA 101:10768-10773.). The protective activity of the methionine aminopeptidase 2 inhibitor in this model was examined to determine if it was linked to the inhibition of the molecular target, MetAP-2. The amount of uninhibited MetAP-2 in white blood cells of animals from all treatment groups was measured after the conclusion of the study, using the MetAP-2 pharmacodynamic assay (Bernier, S. G., et al. (2004) Proc. Natl. Acad. Sci. USA 101: 10768-10773; Bernier, S. G. et al. (2005) J. Cell. Biochem. 95: 1191-1203). In animals orally administered the methionine aminopeptidase 2 inhibitor at 1, 5 and 10 mg/kg, every other day, ≧60% of MetAP-2 in white blood cells was inhibited at the lowest dose, while ≧95% of MetAP-2 was inhibited at 5 and 10 mg/kg, relative to the vehicle-treated group. These results demonstrated that the protective activity of the methionine aminopeptidase 2 inhibitor observed in vivo was linked to the inhibition of MetAP-2 function, and confirmed that the amount of uninhibited MetAP-2 in white blood cells could serve as a pharmacodynamic marker to measure the activity of the methionine aminopeptidase 2 inhibitor in an experimental model of arthritis. Notably, ≧90% MetAP-2 inhibition was also observed after the administration of dexamethasone (dex) (1 mg/kg, orally, every other day). No MetAP-2 inhibition was observed in naive animals treated with dexamethasone for 12 days at 1 mg/kg, every other day, every 4 days or every 6 days, suggesting a potentially novel mechanism of protection from disease for steroids in experimental arthritis (as shown in FIG. 1).

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method of assessing the efficacy of a glucocorticoid treatment for treating a glucocorticoid-associated disease in a subject, the method comprising comparing:
a) the amount of MetAP-2 present in a first sample obtained from the subject prior to administering at least a portion of the glucocorticoid treatment to the subject; and
b) the amount of MetAP-2 present in a second sample obtained from the subject following administration of at least a portion of the glucocorticoid treatment, thereby assessing the efficacy of a glucocorticoid treatment for treating a glucocorticoid-associated disease in a subject.

2. The method of claim 1, wherein a higher amount of MetAP-2 present in the first sample, relative to the second sample, is an indication that the glucocorticoid treatment is efficacious for treating a glucocorticoid-associated disease, and wherein a lower amount of MetAP-2 present in the first sample, relative to the second sample, is an indication that the glucocorticoid treatment is not efficacious for treating a glucocorticoid-associated disease in the subject.

3. The method of claim 1, wherein the amount of MetAP-2 that is determined is the amount of free MetAP-2 protein.

4. The method of claim 1, wherein the amount of MetAP-2 that is determined is the amount of MetAP-2 mRNA.

5. The method of claim 1, wherein determining the amount of MetAP-2 comprises the use of a MetAP-2 inhibitor.

6. The method of claim 5, wherein the MetAP-2 inhibitor is a compound comprising the structure

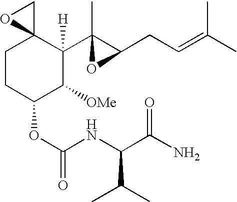

7. The method of claim 5, wherein the MetAP-2 inhibitor is biotinylated.

8. A method of assessing the efficacy of a glucocorticoid treatment for treating a glucocorticoid-associated disease in a subject, the method comprising comparing:
a) the expression and/or activity of MetAP-2 in a first sample obtained from the subject prior to administering at least a portion of the glucocorticoid treatment to the subject; and
b) the expression and/or activity of MetAP-2 in a second sample obtained from the subject following administration of at least a portion of the glucocorticoid treatment, wherein a higher expression and/or activity of MetAP-2 in the first sample, relative to the second sample, is an indication that the glucocorticoid treatment is efficacious for treating a glucocorticoid-associated disease, and wherein a lower expression and/or activity of MetAP-2 in the first sample, relative to the second sample, is an indication that the glucocorticoid treatment is not efficacious for treating a glucocorticoid-associated disease in the subject.

9. The method of claim 8, wherein the expression of MetAP-2 is determined.

10. The method of claim 8, wherein the activity of MetAP-2 is determined.

11. The method of claim 9, wherein the expression of MetAP-2 is determined using a technique selected from the group consisting of quantitative PCR and expression array analysis.

12. The method of claim 10, wherein the activity of MetAP-2 is determined by determining the amount of free methionine in the first and second sample.

13. The method of claim 1 or 8, wherein the sample is a whole blood sample.

14. The method of claim 1 or 8, wherein the sample is selected from the group consisting of tissue and cells.

15. The method of claim 1 or 8, wherein the sample consists of white blood cells.

16. The method of claim 1 or 8, wherein the glucocorticoid is selected from the group consisting of dexamethasone, beclomethasone dipropionate, betamethasone, dipropionatebudesonide, cortisone, deflazacort, flunisolide, fludrocortisone, fluticasone propionate, hydrocortisone, methylprednisolone, prednisolone, and triamcinolone.

17. The method of claim 1 or 8, wherein the glucocorticoid-associated disease is selected from the group consisting of transplant rejection, allergic disease, autoimmune disease, and cancer.

18. The method of claim 17, wherein the autoimmune disease is rheumatoid arthritis.

19. The method of claim 1 or 8, wherein the subject is human.

20. A kit for performing the method of claim 1, comprising (a) a MetAP-2 inhibitor, (b) a detectable antibody that specifically binds to MetAP-2, (c) reagents for isolating whole blood, and (d) instructions for use.

21. A method of treating a subject suffering from a MetAP-2 disease comprising: selecting a subject that would benefit from a decreased amount of MetAP-2; and administering to said subject a therapeutically effective amount of a glucocorticoid, thereby treating a subject suffering from a MetAP-2 disease.

22. A method of treating a subject suffering from a glucocorticoid-associated disease comprising:
selecting a subject that would benefit from treatment with a glucocorticoid; and administering to said subject a therapeutically effective amount of a MetAP-2 inhibitor, thereby treating a subject suffering from a glucocorticoid-associated disease.
